(19) United States
(12) Patent Application Publication
(10) Pub. No.: US 2021/0015552 A1
(43) Pub. Date: Jan. 21, 2021

(54) PATCH ELECTRODE INCLUDING TEMPERATURE SENSING CIRCUIT AND METHODS OF USING SAME

(71) Applicant: St. Jude Medical, Cardiology Division, Inc., St. Paul, MN (US)

(72) Inventors: Timothy G. Curran, St. Paul, MN (US); Lawrence D. Swanson, White Bear Lake, MN (US)

(21) Appl. No.: 16/946,824

(22) Filed: Jul. 8, 2020

Related U.S. Application Data
(60) Provisional application No. 62/875,106, filed on Jul. 17, 2019.

(51) Int. Cl. A61B 18/14; A61B 18/12; A61N 1/04

(52) U.S. Cl. CPC A61B 18/1492; A61B 2018/00577; A61N 1/0492; A61B 18/1206

(57) ABSTRACT

Disclosed herein is an ablation system that includes a catheter electrode, a return patch electrode adapted for attachment to a patient's skin, an ablation generator electrically coupled to the catheter electrode and the return patch electrode and configured to supply ablative energy thereto, and a controller communicatively coupled to the return patch electrode and the ablation generator. The return patch electrode includes a temperature sensing circuit comprising a plurality of discrete temperature sensors arranged across the return patch electrode. The controller is configured to monitor a series resistance of the temperature sensing circuit, and determine that a temperature of the patient's skin exceeds a predetermined threshold based on the series resistance of the temperature sensing circuit

### CROSS-REFERENCE TO RELATED APPLICATIONS

This application claims priority to U.S. provisional patent application Ser. No. 62/875,106, filed Jul. 17, 2019, the disclosure of which is hereby incorporated by reference in its entirety.

### BACKGROUND OF THE DISCLOSURE

### a. Field of the Disclosure

The present disclosure relates generally to methods, systems, and apparatuses for performing an ablation procedure. More particularly, the present disclosure relates to ablation systems and methods for monitoring the temperature at a return patch electrode during an ablation procedure.

### b. Background

Tissue ablation may be used to treat a variety of clinical disorders. For example, tissue ablation may be used to treat cardiac arrhythmias by destroying aberrant pathways that would otherwise conduct abnormal electrical signals to the heart muscle. Several ablation techniques have been developed, including cryoablation, microwave ablation, radio frequency (RF) ablation, and high frequency ultrasound ablation. RF ablation has become increasingly popular for many symptomatic arrhythmias such as AV nodal reentrant tachycardia, AV reciprocating tachycardia, idiopathic ventricular tachycardia, and primary atrial tachycardias. RF ablation is also a common technique for treating disorders of the endometrium and other body tissues including the brain.

A typical RF ablation system includes an RF ablation generator, which feeds current to a catheter containing a conductive tip electrode for contacting targeted tissue. The system is completed by a return path to the RF generator, provided through the patient and a conductive return patch or pad electrode, which is in contact with the patient's skin.

Return electrodes generally have a large patient contact surface area to distribute current density through the return electrode and minimize heating at the return electrode. In some instances, however, current through the return electrode may become concentrated in one or more relatively small areas of the return electrode, resulting in a high current density and creating a potential burn risk. For example, if a portion of the return electrode becomes detached from the patient's skin, the contact area of the electrode decreases resulting in increased current density at the remainder of the return electrode. Additionally, current through the return electrode may become concentrated at certain areas based on the relative density and distribution of muscle, fat, and bone at the site where the return electrode is attached to the patient's skin.

At least some known ablation systems monitor the contact between a return electrode and the patient, for example, by monitoring the impedance at the return electrode. Such systems may calculate a variety of tissue and/or electrode properties (e.g., degree of electrode adhesiveness, average temperature) based on the measured impedance. However, such systems are generally not adapted to detect localized temperature increases or “hot spots” at the return patch electrode.

Accordingly, a need exists for improved systems and methods for monitoring the temperature of a patient's skin at the return patch electrode site.

### SUMMARY OF THE DISCLOSURE

The present disclosure is directed to an ablation system that includes a catheter electrode, a return patch electrode adapted for attachment to a patient's skin, an ablation generator electrically coupled to the catheter electrode and the return patch electrode and configured to supply ablative energy thereto, and a controller communicatively coupled to the return patch electrode and the ablation generator. The return patch electrode includes a temperature sensing circuit comprising a plurality of discrete temperature sensors arranged across the return patch electrode. The controller is configured to monitor a series resistance of the temperature sensing circuit, and determine that a temperature of the patient's skin exceeds a predetermined threshold based on the series resistance of the temperature sensing circuit.

The present disclosure is further directed to a method that includes attaching a return patch electrode to a patient's skin, where the return patch electrode includes a temperature sensing circuit including a plurality of discrete temperature sensors arranged across the return patch electrode. The method further includes monitoring, by a controller communicatively coupled to the return patch electrode, a series resistance of the temperature sensing circuit in response to ablative energy supplied to the patient. The method further includes determining, by the controller, that a temperature of the patient's skin exceeds a predetermined threshold based on the series resistance of the temperature sensing circuit and, upon determining that the temperature of the patient's skin exceeds the predetermined threshold, at least one of throttling, by the controller, the amount of ablative energy supplied to the patient, and generating at least one of an audibly-perceptible alert and a visually-perceptible alert.

The present disclosure is further directed to a return patch electrode for an ablation system. The return patch electrode includes a flexible, electrically conductive substrate having a first side adapted for attachment to a patient's skin, and an opposing, second side, and a temperature sensing circuit coupled to the conductive substrate. The temperature sensing circuit includes a plurality of discrete temperature sensors arranged across the return patch electrode. Each temperature sensor of the plurality of temperature sensors is configured to detect a localized temperature increase that exceeds a pre-determined threshold.

### BRIEF DESCRIPTION OF THE DRAWINGS

FIG. 1 is a schematic and block diagram view of an ablation system.

FIG. 2 is a schematic view of one exemplary embodiment of a return patch electrode suitable for use with the ablation system of FIG. 1.

FIG. 3 is a rear view of another exemplary embodiment of a return patch electrode suitable for use with the ablation system of FIG. 1.

FIG. 4 is a front view of the return patch electrode of FIG. 3.

FIG. 5 is another rear view of the return patch electrode of FIG. 3, in which an insulative layer of the return patch electrode is omitted to illustrate underlying features of the return patch electrode, including a temperature sensing circuit.

FIG. 6 is an enlarged view of the return patch electrode of FIG. 5.

FIG. 7 is an enlarged view of the return patch electrode of FIG. 6.

FIG. 8 is another enlarged view of the return patch electrode of FIG. 6, illustrating a surface mounted thermistor coupled to the temperature sensing circuit.

FIG. 9 is another enlarged view of the return patch electrode of FIG. 6, illustrating a thick-film printed thermistor coupled to the temperature sensing circuit.

FIG. 10 is another enlarged view of the return patch electrode of FIG. 6, illustrating an integrated thermistor coupled to the temperature sensing circuit.

FIG. 11 is a flow diagram illustrating one embodiment of a method of performing an ablation procedure.

### DETAILED DESCRIPTION OF THE DISCLOSURE

The present disclosure is directed to ablation systems and methods and, more particularly, to monitoring the temperature of a patient's skin during an ablation procedure. Embodiments of the systems and methods disclosed herein facilitate monitoring the temperature of a patient's skin and detecting abnormally high temperatures or “hot spots” on the patient's skin at a return patch electrode during the ablation procedure. Upon detecting a “hot spot”, the systems and methods disclosed herein alert an operator of the ablation system and/or throttle the supply of ablative energy to the electrodes. The various approaches described herein may therefore facilitate eliminating or reducing the risk of burning a patient's skin during an ablation procedure.

In particular, embodiments of the present disclosure utilize a return patch electrode that includes a temperature sensing circuit including a plurality of thermistors electrically coupled in series. The thermistors exhibit an increase in resistance as the temperature of the thermistor increases and, in certain embodiments, exhibit a non-linear increase in resistance above a certain temperature. Thus, when any one of the thermistors experiences a relatively large change in temperature (e.g., from a “hot spot” on the return patch electrode), the series resistance of the temperature sensing circuit will significantly increase (e.g., by an order of magnitude or more). Accordingly, by monitoring a series resistance of the temperature sensing circuit, temperature “hot spots” on the return patch electrode (and the patient's skin to which the return patch electrode is connected) can be detected, and appropriate action taken to mitigate the risk of patient burns. Additionally, embodiments of the present disclosure provide a relatively simple, low-cost, reliable “hot spot” detection circuit for use in return patch electrodes in ablation systems. For example, embodiments of the temperature sensing circuits disclosed herein can be implemented as a flex circuit directly on the conductive substrate of a return patch electrode, and require only two additional wires or leads to monitor the temperature sensing circuit.

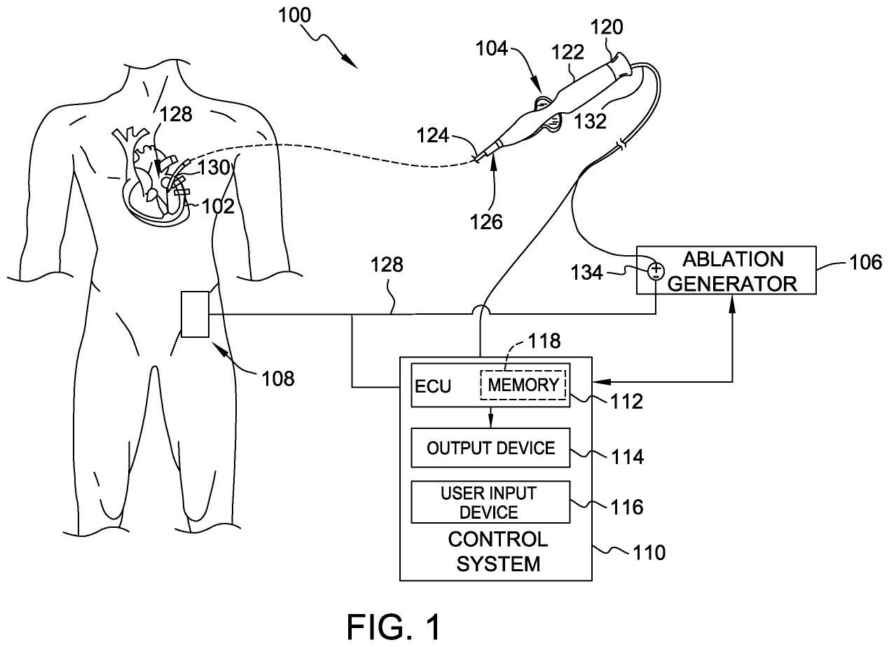

Referring now to the drawings, FIG. 1 illustrates one exemplary embodiment of an ablation system 100 for performing one or more diagnostic and/or therapeutic functions that include components for monitoring the temperature of a return patch electrode (e.g., coupled to a patient's skin) during and/or after an ablation procedure performed on tissue 102 of a patient. In the illustrative embodiment, the tissue 102 is heart or cardiac tissue. It should be understood, however, that the system 100 has equal applicability to ablation procedures on other tissues as well, and is not limited to ablation procedures on cardiac tissue.

The system 100 includes a medical device (such as, for example, a catheter 104), an ablation generator 106, one or more return patch electrodes 108 (also referred to as dispersive or indifferent patch electrodes), and a control system 110 for communicating with and/or controlling one or more components of the ablation system 100. The control system 110 may include, for example and without limitation, a controller or electronic control unit (ECU) 112, an output device 114, user input device 116, and memory 118. In some embodiments, the control system 110 may be implemented in combination with, as part of, or incorporated within other systems and/or sub-systems of the ablation system 100 including, for example and without limitation, the ablation generator 106, imaging systems, mapping systems, navigation systems, and any other system or sub-system of the ablation system 100.

The catheter 104 is provided for examination, diagnosis, and/or treatment of internal body tissues, such as cardiac tissue 102. In an exemplary embodiment, the catheter 104 comprises a radio frequency (RF) ablation catheter. It should be understood, however, that the catheter 104 is not limited to an RF ablation catheter. Rather, in other embodiments, the catheter 104 may comprise an irrigated catheter and/or other types of ablation catheters (e.g., cryoablation, ultrasound, irreversible electroporation, balloon, basket, single electrode, bullet, etc.).

In an exemplary embodiment, the catheter 104 is electrically connected to the ablation generator 106 to allow for the delivery of RF energy. The catheter 104 may include a cable connector or interface 120, a handle 122, a shaft 124 having a proximal end 126 and distal end 128 (as used herein, “proximal” refers to a direction toward the end of catheter 104 near the operator, and “distal” refers to a direction away from the operator and (generally) inside the body of a subject or patient), and one or more electrodes 130 mounted in or on shaft 124 of catheter 104. In an exemplary embodiment, electrode 130 is disposed at or near distal end 128 of shaft 124, with electrode 130 comprising an ablation electrode disposed at the extreme distal end 128 of shaft 124 for contact with cardiac tissue 102. Catheter 104 may further include other conventional components such as, for example and without limitation, sensors, additional electrodes (e.g., ring electrodes) and corresponding conductors or leads, thermocouples, or additional ablation elements, e.g., a high intensity focused ultrasound ablation element and the like.

Connector 120 provides mechanical and electrical connection(s) for cables 132 extending from the ablation generator 106, control system 110, and other systems and/or sub-systems of the ablation system 100. Connector 120 is conventional in the art and is disposed at the proximal end of catheter 104.

Handle 122 provides a location for the operator to hold catheter 104 and may further provide means for steering or guiding shaft 124 within the patient. For example, handle 122 may include means to change the length of a guidewire extending through catheter 104 to distal end 128 of shaft 124 to steer shaft 124. Handle 122 is also conventional in the art and it will be understood that the construction of handle 122 may vary. In another exemplary embodiment, catheter 104 may be robotically driven or controlled. Accordingly, rather than an operator manipulating a handle to steer or guide catheter 104, and shaft 124 thereof, in particular, a robot is used to manipulate catheter 104.

Shaft 124 is generally an elongated, tubular, flexible member configured for movement within the patient. Shaft 124 supports, for example and without limitation, electrode 130, associated conductors, and possibly additional electronics used for signal processing or conditioning. Shaft 124 may also permit transport, delivery and/or removal of fluids (including irrigation fluids, cryogenic ablation fluids, and bodily fluids), medicines, and/or surgical tools or instruments. Shaft 124 may be made from conventional materials such as polyurethane, and defines one or more lumens configured to house and/or transport at least electrical conductors, fluids, or surgical tools. Shaft 124 may be introduced into cardiac tissue 102 through a conventional introducer. Shaft 124 may then be steered or guided within cardiac tissue 102 to a desired location with guidewires or other means known in the art.

Ablation generator 106 generates, delivers, and controls RF energy output by ablation catheter 104 and electrode 130 thereof, in particular. In an exemplary embodiment, ablation generator 106 includes RF ablation signal source 134 configured to generate an ablation signal that is output across a pair of source connectors: a positive polarity connector SOURCE (+), which may be electrically connected to tip electrode 130 of catheter 104; and a negative polarity connector SOURCE (−), which may be electrically connected to the one or more return patch electrodes 108 (e.g., via a conductive lead or cable 136) disposed on the patient's skin.

It should be understood that the term connectors as used herein does not imply a particular type of physical interface mechanism, but is rather broadly contemplated to represent one or more electrical nodes. Source 134 is configured to generate a signal at a predetermined frequency in accordance with one or more user specified parameters (e.g., power, time, etc.) and under the control of various feedback sensing and control circuitry as is known in the art. Source 134 may generate a signal, for example, with a frequency of about 450 kHz to 500 kHz or greater, and may have a power output of up to 50 Watts, up to 75 Watts, up to 100 Watts, up to 150 Watts, up to 200 Watts, or higher. Ablation system 100 may also monitor various parameters associated with the ablation procedure including, for example, impedance, the temperature at the distal tip of the catheter, applied ablation energy, and the position of the catheter, and provide feedback to the operator or another component within system 100 regarding these parameters.

As described in greater detail herein, the return patch electrode 108 includes a temperature sensing circuit configured to monitor a temperature of the patient's skin during an ablation procedure. The temperature sensing circuit is communicatively coupled to the controller 112, which monitors a temperature of the return patch electrode 108 by monitoring one or more parameters of the temperature sensing circuit (e.g., a resistance). If the controller 112 determines that a temperature of the patient's skin exceeds a predetermined threshold, the controller 112 may perform one or more functions to facilitate altering the ablation procedure (e.g., by throttling or terminating the supply of ablative energy) and preventing burns to a patient's skin. In some embodiments, for example, the controller 112 is configured to generate an audibly-perceptible alert and/or a visually-perceptible alert so an operator can throttle or terminate the supply of ablative energy. Additionally or alternatively, the controller 112 can be configured to automatically throttle or terminate the supply of ablative energy to the catheter electrode 130 when the controller 112 determines that a temperature of the patient's skin exceeds a predetermined threshold.

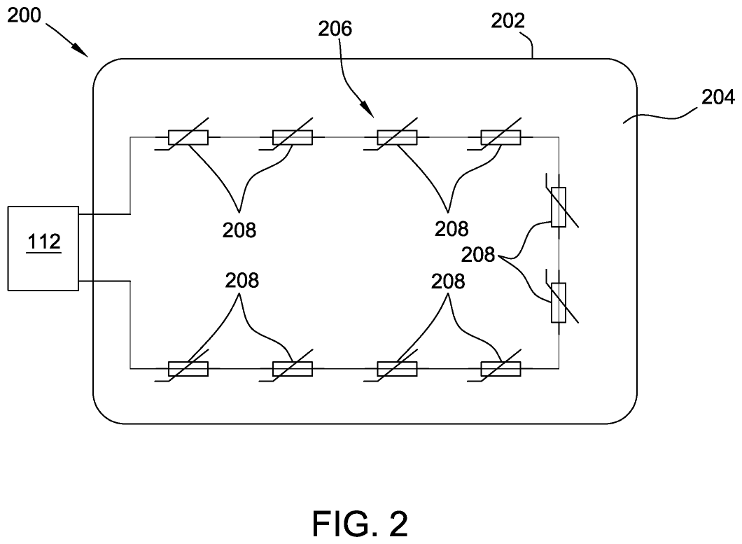

FIG. 2 is a schematic view of an exemplary embodiment of a return patch electrode 200 suitable for use in the ablation system 100 of FIG. 1. In the illustrated embodiment, the return patch electrode 200 includes a flexible, electrically conductive substrate 202 having a first side (not shown in FIG. 2) adapted for attachment to a patient's skin, and an opposing, second side 204. The conductive substrate 202 is sufficiently flexible such that the patch electrode 200 is capable of conforming to a patient's skin to facilitate electrical contact between the electrode and the patient's skin. The conductive substrate 202 is also electrically conductive to enable conduction of electrical ablative energy (e.g., RF energy) through the patient's skin. The conductive substrate 202 can be constructed from any suitably electrically conductive, flexible substrate that enables the return patch electrode 200 to function as described herein, including, for example and without limitation, aluminum alloy foils and carbon foils. Although not shown in FIG. 2, the conductive substrate 202 also includes an electrical lead or cable (e.g., electrical lead 136, shown in FIG. 1) electrically and physically coupled to the conductive substrate 202 for electrically coupling the return patch electrode 200 to the ablation generator 106.

In the illustrated embodiment, the return patch electrode 200 is a single piece electrode—i.e., the conductive substrate 202 is constructed of a single, continuous substrate (e.g., conductive foil). In other words, the return patch electrode 200 of the illustrated embodiment is not a “split” return patch electrode, in which the electrode is split or separated into multiple electrode segments or pieces that are electrically isolated from one another and rely on conductance through the patient to complete an electrical circuit between the separate electrode parts. In other embodiments, the return patch electrode 200 may have a “split” electrode construction.

The return patch electrode 200 further includes a temperature sensing circuit 206 coupled to the conductive substrate 202. The temperature sensing circuit 206 is communicatively coupled to the controller 112, and is configured to detect localized temperature increases or “hot spots” on a patient's skin during an ablation procedure. The temperature sensing circuit 206 includes a plurality of discrete temperature sensors 208 arranged across the return patch electrode 200. Each temperature sensor 208 is configured to detect a localized temperature increase that exceeds a pre-determined temperature threshold. The controller 112 monitors one or more temperature-dependent parameters of the temperature sensing circuit 206 (e.g., a resistance). When one or more of the temperature sensors 208 detects a localized temperature increase above the pre-determined threshold, the controller 112 detects a change in the one or more temperature-dependent parameters of the temperature sensing circuit 206, and determines that the pre-determined temperature threshold has been exceeded.

In the illustrated embodiment, the temperature sensing circuit 206 includes 10 temperature sensors, although the temperature sensing circuit 206 may include any suitable number of temperature sensors that enables the ablation system 100 to function as described herein. For example, the temperature sensing circuit 206 can include between 2 temperature sensors and 40 temperature sensors, between 2 temperature sensors and 30 temperature sensors, between 5 temperature sensors and 40 temperature sensors, between 2 temperature sensors and 20 temperature sensors, between 4 temperature sensors and 30 temperature sensors, and between 4 temperature sensors and 20 temperature sensors. In other embodiments, the temperature sensing circuit 206 can include fewer than 2 temperature sensors, or more than 40 temperature sensors.

In the exemplary embodiment, the discrete temperature sensors 208 are resistors and, more specifically, thermistors 208 that are electrically coupled in series to form the temperature sensing circuit 206. Thus, as the temperature of the return patch electrode 200 changes, each of the thermistors 208 will undergo a corresponding change in resistance, causing the series resistance of the temperature sensing circuit 206 to change. In this embodiment, the controller 112 is configured to monitor the temperature of a patient's skin by monitoring the series resistance of the temperature sensing circuit 206. If the controller 112 detects that the series resistance of the temperature sensing circuit 206 deviates beyond a predetermined threshold, the controller 112 may perform one or more functions to facilitate adjusting or terminating the ablation procedure to prevent burns to a patient's skin, such as generating an alert and/or automatically throttling or terminating the supply of ablative (e.g., RF) energy, as described herein.

The thermistors 208 may generally include any suitable thermistor that enables the ablation system 100 to function as described herein. In some embodiments, for example, the thermistors are positive temperature coefficient (PTC) thermistors. That is, the resistance of the thermistors increases as the temperature of the thermistors increases. Further, in some embodiments, one or more of the thermistors may have an associated temperature threshold or “Curie point” at which the temperature response of the thermistor resistance transitions from a linear response to a non-linear response. In some embodiments, for example, the resistance of the thermistor exhibits a positive, exponential response to increases in temperature above the Curie point such that, when the temperature of the thermistor exceeds the Curie point, the resistance of the thermistors rapidly increases. In some embodiments, the transition between the linear response and the non-linear response is associated with a material phase transition of the thermistor between a first state, in which the thermistor exhibits ferroelectric (i.e., electrically conductive) properties, and a second state, in which the thermistor exhibits paraelectric (i.e., electrically insulating) properties.

The thermistors may be implemented in the temperature sensing circuit 206 using any suitable circuit components and techniques including, for example and without limitation, surface mounted thermistors, thick-film printed thermistors, and integrated thermistors (i.e., thermistors formed integrally with the temperature sensing circuit 206 using, for example integrated circuit (IC) techniques). Further, the construction of the thermistors can be selected to achieve a desired Curie point or transition temperature. In some embodiments, for example, the thermistors have a Curie point that corresponds to the pre-determined temperature threshold above which the controller 112 performs one or more functions to facilitate altering the ablation procedure. In some embodiments, for example, the thermistors have a Curie point of between 30° C. and 50° C., between 30° C. and 40° C., or between 40° C. and 50° C. In other embodiments, the thermistors may have any suitable Curie point that enables that ablation system 100 to function as described herein. The illustrated embodiment includes 10 thermistors electrically coupled in series, although the temperature sensing circuit 206 may include any suitable number of thermistors that enables the ablation system 100 to function as described herein, including any number of thermistors within the numerical ranges of temperature sensors disclosed herein.

Use of PTC thermistors that exhibit a non-linear response to temperature increases above a certain temperature or Curie point can facilitate quickly and accurately detecting hot spots at the return patch electrode 200. For example, when the temperature of one or more of the PCT thermistors exceeds the Curie point, the resistance of the one or more thermistors will significantly increase (e.g., by an order of magnitude or more), causing the series resistance of the temperature sensing circuit 206 to likewise significantly increase (e.g., by an order of magnitude or more). The large change in series resistance of the temperature sensing circuit 206 can be readily detected by the controller 112, which can then determine that the temperature of the return patch electrode 200 has exceeded the pre-determined temperature threshold. Based on this determination, the controller 112 can perform one or more functions to facilitate altering the ablation procedure to prevent burns to a patient's skin, including generating an audibly-perceptible alert and/or a visually-perceptible alert, and automatically throttling or terminating the supply of ablative energy to the catheter electrode 130.

The pre-determined temperature threshold may generally correspond to a temperature below which there is little or no risk of patient burn, and above which there is appreciable or unacceptable risk of patient burn. In some embodiments, for example, the predetermined temperature threshold is between 30° C. and 50° C., between 30° C. and 40° C., or between 40° C. and 50° C. In other embodiments, the predetermined temperature threshold may be any suitable temperature that enables the ablation system 100 to function as described herein.

As noted above, in the exemplary embodiment, the controller 112 is configured to monitor the temperature of a patient's skin by monitoring the series resistance of the temperature sensing circuit 206. The controller 112 determines that a temperature of the patient's skin exceeds a predetermined temperature threshold based on the measured series resistance of the temperature sensing circuit 206. For example, if the controller 112 detects that the series resistance of the temperature sensing circuit 206 exceeds a predetermined resistance threshold, the controller 112 may determine that a temperature of the patient's skin exceeds the predetermined temperature threshold, and perform one or more functions to facilitate altering the ablation procedure and preventing burns to a patient's skin. In some embodiments, for example, the controller 112 is configured to generate at least one of an audibly-perceptible alert and a visually-perceptible alert (e.g., via output device 114) upon determining that the temperature of the patient's skin exceeds the predetermined threshold to alert an operator of the ablation system 100.

Additionally or alternatively, the controller 112 can be configured to automatically throttle or terminate the supply of ablative energy to the catheter electrode 130 upon determining that the temperature of the patient's skin exceeds the predetermined threshold. In some embodiments, for example, the controller 112 is configured to automatically terminate or shut off the supply of ablative energy to the catheter electrode 130 upon determining that the temperature of the patient's skin exceeds the predetermined threshold.

In other embodiments, the controller 112 is configured to automatically throttle the supply of ablative energy to the catheter electrode 130 to a reduced, non-zero power level upon determining that the temperature of the patient's skin exceeds the predetermined threshold. In some embodiments, for example, the controller 112 is configured to throttle the supply of ablative energy to the catheter electrode 130 to a first reduced power level upon determining that the temperature of the patient's skin exceeds a first predetermined temperature threshold. In such embodiments, the controller 112 may be further configured to throttle the supply of ablative energy to the catheter electrode 130 to a second reduced power level less than the first reduced power level upon determining that the temperature of the patient's skin exceeds a second predetermined temperature threshold greater than the first predetermined threshold. The first reduced power level is generally a non-zero power level less than the standard or typical operating power of the ablation generator 106 used under normal operating conditions. The second reduced power level may be a zero or non-zero power level. In embodiments where the second reduced power level is a zero power level (i.e., a power output of zero), the controller 112 is configured to terminate the supply of ablative energy to the catheter electrode 130 upon determining that the temperature of the patient's skin exceeds the second predetermined threshold.

As noted above, the controller 112 in certain embodiments monitors the resistance of the temperature sensing circuit 206 to determine if a patient's skin exceeds a predetermined temperature threshold. In some embodiments, for example, the controller 112 compares a measured series resistance of the temperature sensing circuit 206 to a baseline series resistance of the temperature sensing circuit 206 to determine whether the temperature of a patient's skin exceeds the predetermined threshold. In such embodiments, the controller 112 may determine that a temperature of the patient's skin exceeds the predetermined threshold when the measured series resistance of the temperature sensing circuit 206 exceeds the baseline series resistance by a certain amount. For example, the controller 112 may determine that a temperature of the patient's skin exceeds the predetermined threshold when the measured series resistance of the temperature sensing circuit 206 is at least 10%, at least 25%, at least 50%, at least 75%, at least 100%, at least 150%, at least 200%, at least 300%, at least 400%, or at least 500% greater than the baseline series resistance. In other embodiments, percentage changes in the measured series resistance of less than 10% or greater than 500% may be used to determine that a temperature of the patient's skin exceeds the predetermined threshold.

The baseline resistance of the temperature sensing circuit generally corresponds to the resistance of the temperature sensing circuit under normal operating conditions (i.e., in the absence of temperature “hot spots” on a patient's skin). The baseline resistance may be measured and established under controlled environmental conditions (e.g., at room temperature or an average skin temperature of a patient), and stored in the memory 118 of controller 112. Additionally or alternatively, the baseline resistance of the temperature sensing circuit 206 may be a dynamic baseline resistance, and determined or established at the beginning of each ablation procedure (i.e., prior to ablation energy being supplied to the electrodes). In some embodiments, for example, the controller 112 is configured to determine the baseline series resistance by measuring a series resistance of the temperature sensing circuit 206 subsequent to the return patch electrode 200 being attached to a patient's skin, and storing the measured series resistance as the baseline series resistance in the memory 118. In other embodiments, the baseline series resistance may be established using any suitable techniques that enables the ablation system 100 to function as described herein.

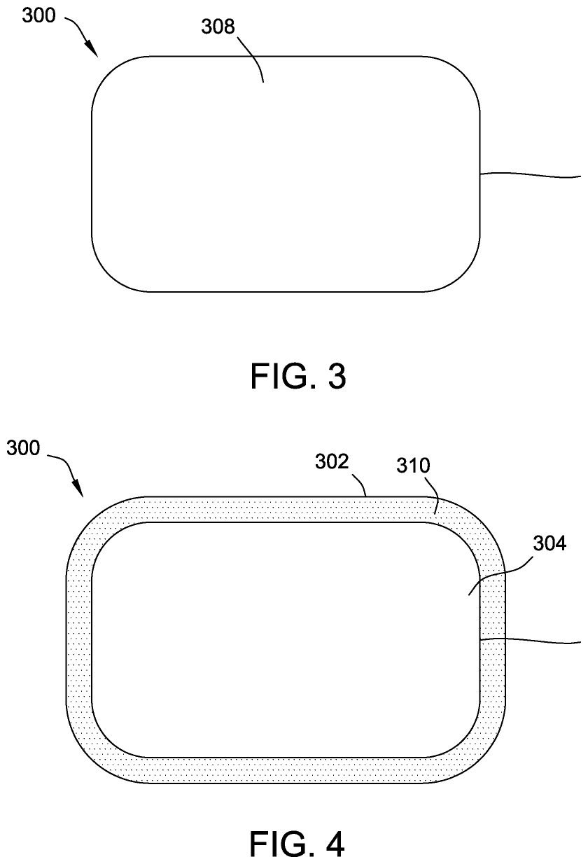

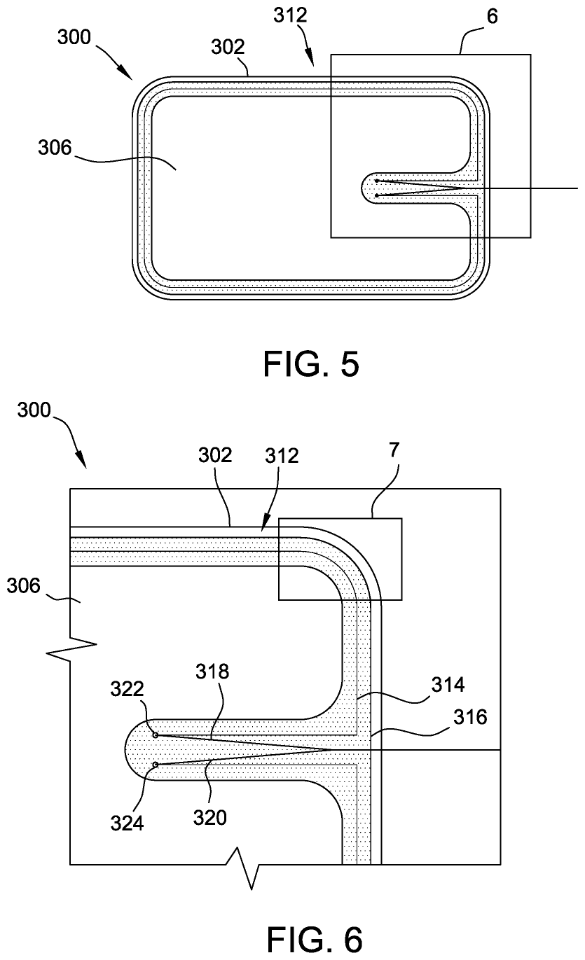

FIG. 3 is a rear view of another exemplary embodiment of a return patch electrode 300 suitable for use with the ablation system 100 of FIG. 1. FIG. 4 is a front view of the return patch electrode 300, and FIG. 5 is another rear view of the return patch electrode 300 with an electrically insulative layer of the return patch electrode 300 omitted to illustrate underlying features of the return patch electrode 300.

As shown in FIGS. 3-5, the return patch electrode 300 includes a flexible, electrically conductive substrate 302 having a first side 304 adapted for attachment to a patient's skin, and an opposing, second side 306, and an electrically insulative layer 308 coupled to the second side 306. The electrically conductive substrate 302 is sufficiently flexible such that the patch electrode 300 is capable of conforming to a patient's skin to facilitate electrical contact between the patch electrode 300 and the patient's skin. The conductive substrate 302 is also electrically conductive to enable conduction of electrical ablative energy (e.g., RF energy) through the patient's skin. The conductive substrate 302 can be constructed from any suitably electrically conductive, flexible substrate that enables the return patch electrode 300 to function as described herein, including, for example and without limitation, aluminum alloy foils and carbon foils. The insulative layer 308 is likewise sufficiently flexible such that the patch electrode 300 is capable of conforming to a patient's skin. The insulative layer 308 is electrically insulating, and can be constructed from any suitably electrically insulative, flexible substrate that enables the return patch electrode 300 to function as described herein, including, for example and without limitation, insulating foams.

In the illustrated embodiment, the return patch electrode 300 also includes electrically conductive adhesive or gel 310 disposed on the first side 304 of the electrically conductive substrate 302 to facilitate attaching the return patch electrode 300 to a patient's skin. The electrically conductive gel 310 is disposed around an outer perimeter of the return patch electrode 300 in the illustrated embodiment, though it should be understood that the electrically conductive gel 310 may be arranged on the electrically conductive substrate 302 in any suitable manner that enables the return patch electrode 300 to function as described herein. The electrically conductive gel 310 may include any suitable electrically conductive gel that enables the return patch electrode 300 to function as described herein, including, for example and without limitation, acrylic-based adhesives or gels.

The return patch electrode 300 also includes a temperature sensing circuit 312 coupled to the electrically conductive substrate 302. The temperature sensing circuit 312 can have substantially the same construction and operate in substantially the same manner as the temperature sensing circuit 206 described above with reference to FIG. 2. For example, the temperature sensing circuit 312 includes a plurality of discrete temperature sensors (not labeled in FIGS. 3-5) arranged across the return patch electrode 300. Each of the temperature sensors is configured to detect a localized temperature increase that exceeds a pre-determined temperature threshold to facilitate detecting hot spots on a patient's skin. In this embodiment, the temperature sensing circuit 312 is thermally coupled to the second side 306 of the electrically conductive substrate 302, and is interposed between the electrically conductive substrate 302 and the electrically insulative layer 308.

In this embodiment, the temperature sensing circuit 312 and temperature sensors thereof are disposed around an outer perimeter of the return patch electrode 300 in the shape of a rectangle. It should be understood that, in other embodiments, the temperature sensing circuit 312 and temperature sensors thereof may be arranged on the return patch electrode 300 in any suitable manner that enables the return patch electrode 300 to function as described herein, including, for example and without limitation, circular patterns, square patterns, rectangular patterns, serpentine patterns, circuitous patterns, and combinations thereof.

FIG. 6 is an enlarged view of the return patch electrode 300 of FIG. 5. As shown in FIG. 6, the temperature sensing circuit 312 of this embodiment is constructed as a flexible circuit on the second side 306 of the electrically conductive substrate 302, and includes a conductive trace 314 disposed on a suitably insulative substrate 316. The conductive trace 314 is constructed of a suitably electrically conductive material, including, for example and without limitation, copper, aluminum, and combinations or alloys thereof The insulative substrate 316 electrically insulates the conductive trace 314 from the electrically conductive substrate 302 of the return patch electrode 300, and is constructed of a suitably electrically insulative material, including, for example and without limitation, a polyimide film.

The return patch electrode 300 includes two lead wires or cables 318, 320 electrically coupled thereto. A first end of each lead wire 318, 320 is connected to a respective terminal end 322, 324 of the temperature sensing circuit 312. The other end of each lead wire 318, 320 (not shown in FIG. 6) is connected to the controller 112 to provide communication between the return patch electrode 300 and the controller 112, for example, to allow the controller 112 to interrogate the temperature sensing circuit 312 and monitor or measure a series resistance of the temperature sensing circuit 312.

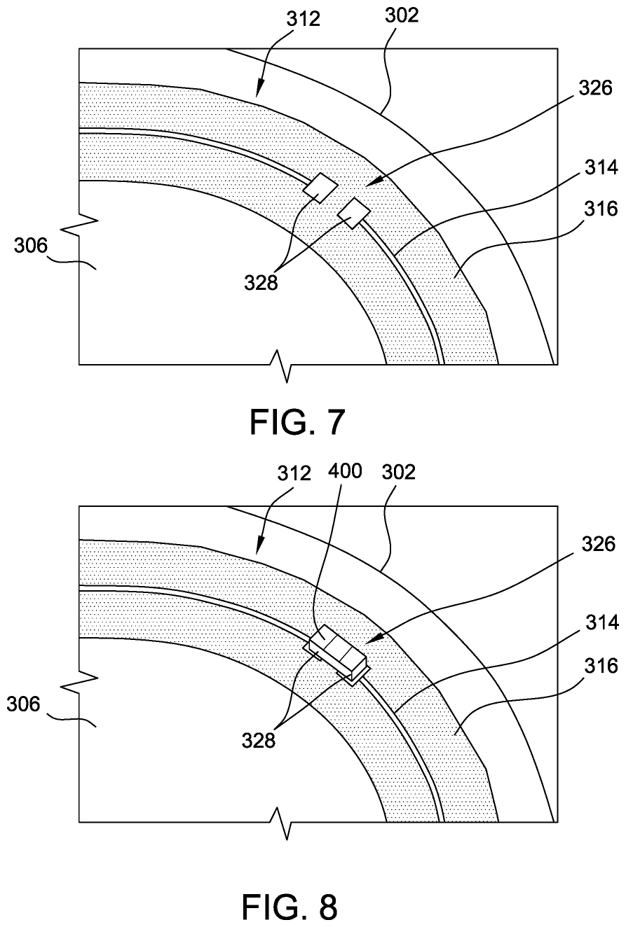

FIG. 7 is an enlarged view of the return patch electrode of FIG. 6. As shown in FIG. 7, the temperature sensing circuit 312 of this embodiment includes a plurality of conductive pad pairs 326 (one shown in FIG. 7) for electrically connecting suitable temperature sensors (e.g., thermistors) to the temperature sensing circuit 312. Each conductive pad pair 326 includes two electrically conductive pads 328 spaced apart and electrically insulated from one another. In this embodiment, suitable thermistors are electrically coupled to the temperature sensing circuit 312 via conductive pads 328, and function as the temperature sensors, as described herein. The thermistors may be implemented in the temperature sensing circuit 312 using any suitable circuit components and techniques including, for example and without limitation, surface mounted thermistors, thick-film printed thermistors, and integrated thermistors (i.e., thermistors formed integrally with the temperature sensing circuit 312 using, for example IC techniques).

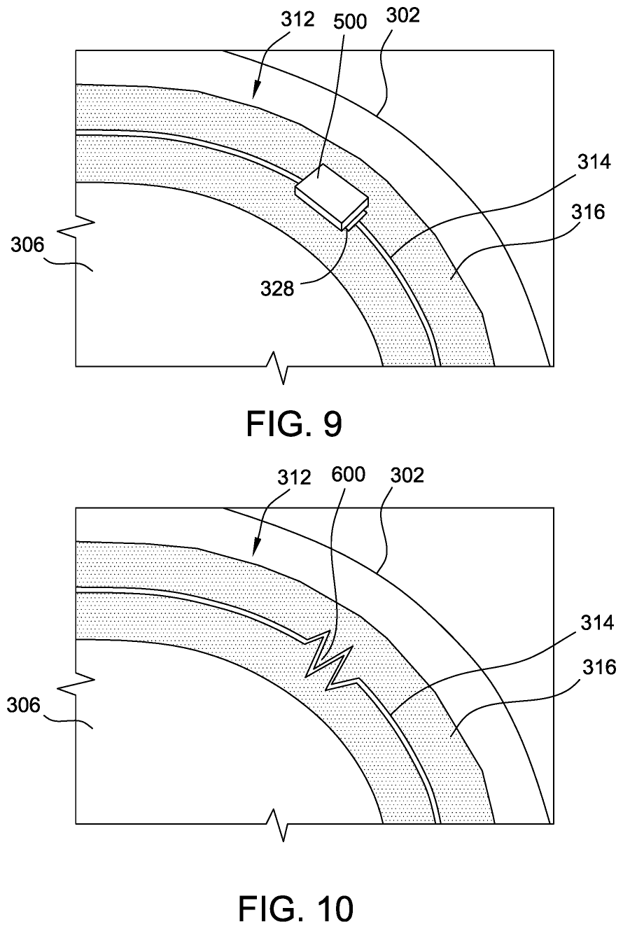

FIG. 8, for example, illustrates the temperature sensing circuit 312 with a surface mounted thermistor 400 coupled thereto via the pair of conductive pads 328. FIG. 9 illustrates the temperature sensing circuit 312 with a thick-film printed thermistor 500 coupled thereto via the pair of conductive pads 328. FIG. 10 schematically illustrates the temperature sensing circuit 312 with an integrated thermistor 600 coupled thereto. In this embodiment, the integrated thermistor 600 is formed integrally with the temperature sensing circuit 312 (e.g., using suitable printed circuit techniques), and the conductive pad pairs 326 are omitted from the temperature sensing circuit 312.

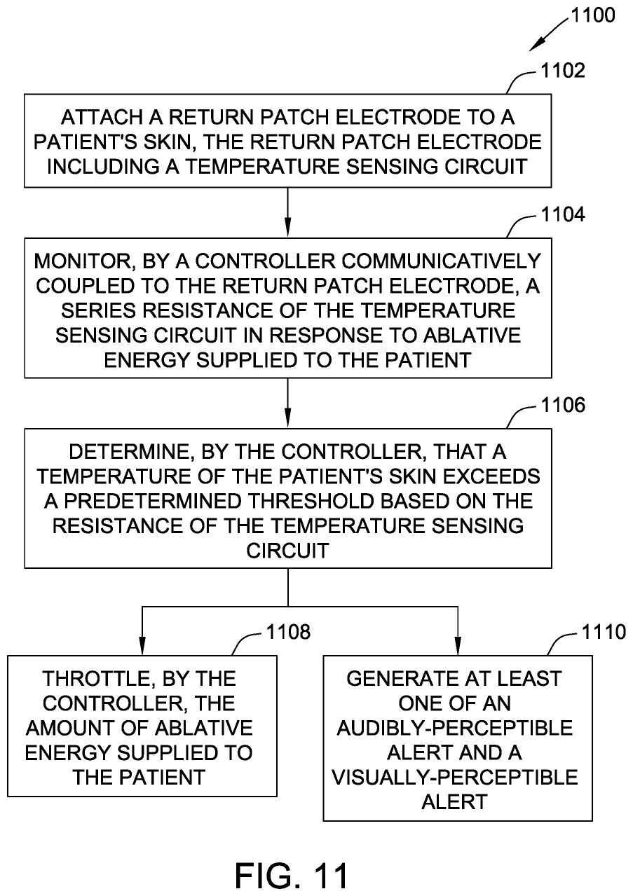

FIG. 11 is a flow diagram illustrating one embodiment of a method 1100 of performing an ablation procedure using an ablation system, such as the ablation system 100 shown in FIG. 1. In the illustrated embodiment, the method 1100 includes attaching 1102 a return patch electrode (e.g., return patch electrodes 200, 300) to a patient's skin. The return patch electrode includes a temperature sensing circuit (e.g., temperature sensing circuits 206, 312) that includes a plurality of discrete temperature sensors arranged across the return patch electrode. The method 1100 further includes monitoring 1104, by a controller (e.g., controller 112) communicatively coupled to the return patch electrode, a series resistance of the temperature sensing circuit in response to ablative energy supplied to the patient. The method 1100 further includes determining 1106, by the controller, that a temperature of the patient's skin exceeds a predetermined threshold based on the resistance of the temperature sensing circuit and, upon determining that the temperature of the patient's skin exceeds the predetermined threshold, at least one of throttling 1108, by the controller, the amount of ablative energy supplied to the patient, and generating 1110 at least one of an audibly-perceptible alert and a visually-perceptible alert.

Although certain steps of the example method are numbered, such numbering does not indicate that the steps must be performed in the order listed. Thus, particular steps need not be performed in the exact order they are presented, unless the description thereof specifically require such order. The steps may be performed in the order listed, or in another suitable order.

Although the embodiments and examples disclosed herein have been described with reference to particular embodiments, it is to be understood that these embodiments and examples are merely illustrative of the principles and applications of the present disclosure. It is therefore to be understood that numerous modifications can be made to the illustrative embodiments and examples and that other arrangements can be devised without departing from the spirit and scope of the present disclosure as defined by the claims. Thus, it is intended that the present application cover the modifications and variations of these embodiments and their equivalents.

This written description uses examples to disclose the invention, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An ablation system comprising:
a catheter electrode;
a return patch electrode adapted for attachment to a patient's skin, the return patch electrode comprising a temperature sensing circuit comprising a plurality of discrete temperature sensors arranged across the return patch electrode;
an ablation generator electrically coupled to the catheter electrode and the return patch electrode and configured to supply ablative energy thereto; and
a controller communicatively coupled to the return patch electrode and the ablation generator, wherein the controller is configured to:
monitor a series resistance of the temperature sensing circuit; and
determine that a temperature of the patient's skin exceeds a predetermined threshold based on the series resistance of the temperature sensing circuit.

2. The ablation system of claim 1, wherein the controller is further configured to terminate the supply of ablative energy to the catheter electrode upon determining that the temperature of the patient's skin exceeds the predetermined threshold.

3. The ablation system of claim 1, wherein the controller is further configured to generate at least one of an audibly-perceptible alert and a visually-perceptible alert upon determining that the temperature of the patient's skin exceeds the predetermined threshold.

4. The ablation system of claim 1, wherein the predetermined threshold is a first predetermined threshold, and wherein the controller is further configured to:
throttle the supply of ablative energy to the catheter electrode to a first reduced power level upon determining that the temperature of the patient's skin exceeds the first predetermined threshold; and
throttle the supply of ablative energy to the catheter electrode to a second reduced power level less than the first reduced power level upon determining that the temperature of the patient's skin exceeds a second predetermined threshold greater than the first predetermined threshold.

5. The ablation system of claim 4, wherein the second reduced power level corresponds to a power output of zero such that the controller is configured to terminate the supply of ablative energy to the catheter electrode upon determining that the temperature of the patient's skin exceeds the second predetermined threshold.

6. The ablation system of claim 1, wherein the temperature sensing circuit has a baseline series resistance, and wherein the controller is configured to determine that a temperature of the patient's skin exceeds a predetermined threshold when a measured series resistance of the temperature sensing circuit is at least 25% greater than the baseline series resistance.

7. The ablation system of claim 6, wherein the controller is further configured to determine the baseline series resistance by:
measuring a series resistance of the temperature sensing circuit subsequent to the return patch electrode being attached to a patient's skin; and
storing the measured series resistance as the baseline series resistance in a memory of the controller.

8. The ablation system of claim 1, wherein the ablation generator is a radiofrequency ablation generator having a power output of up to 150 watts.

9. The ablation system of claim 1, wherein the plurality of discrete temperature sensors comprises a plurality of thermistors electrically coupled in series.

10. The ablation system of claim 9, wherein the plurality of thermistors comprises a plurality of positive temperature coefficient (PTC) thermistors.

11. The ablation system of claim 10, wherein each PTC thermistor of the plurality of PTC thermistors has a Curie point of between 40° C. and 50° C.

12. The ablation system of claim 9, wherein the plurality of thermistors comprises a plurality of surface mounted thermistors.

13. The ablation system of claim 9, wherein the plurality of thermistors comprises a plurality of thick-film printed thermistors.

14. The ablation system of claim 1, wherein the return patch electrode comprises a flexible, electrically conductive substrate and an electrically insulative layer coupled to the electrically conductive substrate, wherein the temperature sensing circuit is interposed between the electrically conductive substrate and the electrically insulative layer.

15. The ablation system of claim 1, wherein the return patch electrode comprises a flexible, electrically conductive substrate having a first side adapted for attachment to a patient's skin, and an opposing, second side, wherein the temperature sensing circuit is coupled to the second side of the electrically conductive substrate.

16. The ablation system of claim 1, wherein the temperature sensing circuit comprises between 4 and 40 temperature sensors.

17. A method comprising:
attaching a return patch electrode to a patient's skin, wherein the return patch electrode includes a temperature sensing circuit that includes a plurality of discrete temperature sensors arranged across the return patch electrode;
monitoring, by a controller communicatively coupled to the return patch electrode, a series resistance of the temperature sensing circuit in response to ablative energy supplied to the patient;
determining, by the controller, that a temperature of the patient's skin exceeds a predetermined threshold based on the series resistance of the temperature sensing circuit; and
upon determining that the temperature of the patient's skin exceeds the predetermined threshold, at least one of:
throttling, by the controller, the amount of ablative energy supplied to the patient; and
generating at least one of an audibly-perceptible alert and a visually-perceptible alert.

18. The method of claim 17, wherein the predetermined threshold is a first predetermined threshold, and wherein the method comprises:
throttling, by the controller, the amount of ablative energy supplied to the patient to a first reduced power level upon determining that the temperature of the patient's skin exceeds the first predetermined threshold; and
throttling, by the controller, the amount of ablative energy supplied to the patient to a second reduced power level upon determining that the temperature of the patient's skin exceeds a second predetermined threshold greater than the first predetermined threshold.

19. The method of claim 17, wherein throttling the amount of ablative energy supplied to the patient to a second reduced power level comprises terminating the supply of ablative energy.

20. The method of claim 17, wherein determining that a temperature of the patient's skin exceeds a predetermined threshold comprises determining that the monitored series resistance of the temperature sensing circuit is at least 25% greater than a baseline series resistance of the temperature sensing circuit.

21. The method of claim 20, further comprising determining the baseline series resistance by:
measuring, by the controller, a series resistance of the temperature sensing circuit subsequent to the return patch electrode being attached to the patient's skin; and
storing the measured series resistance as the baseline series resistance in a memory of the controller.

22. A return patch electrode for an ablation system, said return patch electrode comprising:
a flexible, electrically conductive substrate having a first side adapted for attachment to a patient's skin, and an opposing, second side; and
a temperature sensing circuit coupled to the conductive substrate, the temperature sensing circuit comprising a plurality of discrete temperature sensors arranged across the return patch electrode, each temperature sensor of the plurality of temperature sensors configured to detect a localized temperature increase that exceeds a pre-determined threshold.
